(19) United States
(12) Patent Application Publication
(10) Pub. No.: US 2012/0252829 A1
(43) Pub. Date: Oct. 4, 2012

(54) TIVOZANIB AND CAPECITABINE COMBINATION THERAPY

(76) Inventors: Jie Lin, West Roxbury, MA (US); Jeno Gyuris, Lincoln, MA (US); Feng Jiang, Natick, MA (US); Guangmu Li, Acton, MA (US); Xiaojian Sun, Goleta, CA (US)

(21) Appl. No.: 13/436,021

(22) Filed: Mar. 30, 2012

Related U.S. Application Data
(60) Provisional application No. 61/470,791, filed on Apr. 1, 2011.

(51) Int. Cl. A61K 31/513; A61P 35/00

(52) U.S. Cl. 514/274

(57) ABSTRACT

A method of treating a tumor in a subject using a combination of tivozanib and capecitabine is disclosed.

### CROSS-REFERENCE TO RELATED APPLICATIONS

This application claims the benefit of and priority to U.S. provisional patent application Ser. No. 61/470,791, filed Apr. 1, 2011, the entire content of which is incorporated herein by reference.

### FIELD OF THE INVENTION

The field of the invention is medicine, oncology, tyrosine kinase inhibitors, VEGF receptor inhibitors, DNA synthesis inhibitors, pyrimidine antagonists, antimetabolites, and pharmaceuticals.

### BACKGROUND

Tivozanib (also known as AV-951 and KRN951) is a potent and selective small-molecule inhibitor of VEGF receptors 1, 2 and 3. Tivozanib exhibits picomolar inhibitory activity against all three receptors, and it exhibits antitumor activity in preclinical models (Nakamura et al., 2006, Cancer Res. 66:9134-9142). Tivozanib has yielded positive results in a 272-patient Phase 2 clinical trial (Bhargava et al., 2009, ASCO Annual Meeting Proceedings, Vol 27, No. 15s, Abstract No. 5032). The most common side effects associated with tivozanib treatment in Phase 1, Phase 2 and Phase 3 clinical trials are hypertension and dysphonia.

Capecitabine is a systemic pro-drug of 5′-deoxy-5-fluorouridine, which is metabolized to form 5-fluorouracil (5-FU). 5-FU is a pyrimidine antagonist, inhibiting DNA synthesis and is recognized as a member of the antimetabolite drug family. Several antimetabolites are known, including capecitabine, 5-fluorouracil, mercaptopurine, cladribine, and cytarabine. Capecitabine (XOLEDA) and 5-fluorouracil have received FDA marketing approval as monotherapies for metastatic breast cancer and colorectal cancer. As a monotherapy, capecitabine is typically administered orally twice a day for 14 days, followed by a 7-day rest period. The side effects of capecitabine can be severe, including hand-and-foot syndrome (pain, swelling or redness of your hands or feet), diarrhea, nausea, vomiting, dehydration, sores in the mouth and throat, stomach area pain, tiredness or weakness, fever, and joint and muscle pain.

With any drug, optimal dosage involves balancing the desired therapeutic effect against unwanted side effects, i.e., drug toxicities. The dosage range that yields a therapeutic effect with an acceptable side effect profile is known as the therapeutic window. When two drugs are used in combination, the situation with respect to therapeutic window can become complicated and unpredictable. The therapeutic effects of the two drugs can be non-additive, additive or synergistic. Similarly, the unwanted side effects, i.e., the drug toxicities, can be non-additive, additive or synergistic.

Previous studies combining anti-VEGF pathway agents with targeted therapies or chemotherapies have resulted in mixed clinical results, underscoring the unpredictability of combining anti-VEGF agents with other therapies. Thus, there is still an unmet need for effective and safe combination therapies with VEGF tyrosine kinase inhibitors.

### SUMMARY

The invention is based, in part, on murine tumor model data demonstrating that tivozanib, a VEGF tyrosine kinase inhibitor, can be administered in combination with capecitabine, a pyrimidine antagonist. As disclosed herein, when administered in combination, tivozanib and capecitabine may be administered at reduced doses, suggesting synergistic activity when the two drugs are used in combination.

The invention provides a method of treating a tumor in a human patient, comprising co-administering to the patient: (a) a dose of 0.5-2.0 mg tivozanib per day; and (b) a dose of 400-2000 mg/m² capecitabine twice daily. In some embodiments, tivozanib is administered on a repeating schedule of one dose per day for three weeks, followed by a period without tivozanib administration. In other embodiments, capecitabine is administered on a repeating schedule of one dose twice daily for two weeks, followed by a period without capecitabine administration. Alternatively, capecitabine may be administered on a repeating schedule of one dose twice daily for one week, followed by a one-week period without capecitabine administration. The method may be used for treating various types of solid tumors, e.g., breast tumors and colorectal tumors. In certain embodiments, the tumor is a breast tumor.

### DESCRIPTION OF THE DRAWINGS

The invention can be more completely understood with reference to the following drawings.

FIG. 1 is a graph showing the results from an experiment to measure the tumor inhibitory activity of tivozanib (alone), capecitabine (alone), and tivozanib+capecitabine (co-administered). The following is depicted in the graph: vehicle (, Veh), tivozanib (▪, Tivozanib administered at a dosage of 5 mg per kg), capecitabine (▴, Capecitabine administered at a dosage of 300 mg per kg), and tivozanib+capecitabine (▾, Tivozanib+Capecitabine co-administered at a dosage of 5 mg per kg and 300 mg per kg, respectively).

FIG. 2A is a graph showing the results from an experiment to measure the respective anti-proliferation activity of capecitabine (alone) and tivozanib+capecitabine (co-administered). The capecitabine response is measured after 2, 5 and 9 days of treatment and after two different dosage cycles ((1) 14 days-on, 2 days-off; (2) 14 days-on, 5 days-off). The co-administered tivozanib+capecitabine response is measured after 35 days.

FIG. 2B are photographs of the tumor cells showing the results from an experiment to measure the anti-proliferation activity of capecitabine (alone) and tivozanib+capecitabine (co-administered) as shown in FIG. 2A. The capecitabine response is measured after 2 days (capecitabine 2d) and after a dosage cycle (capecitabine 14d-on and 5d-off). The co-administered tivozanib+capecitabine response (capecitabine+tivozanib 35d) is measured after 35 days.

FIG. 3A is a graph showing the results from an experiment to measure the tumor inhibitory activity of tivozanib+capecitabine (co-administered) with less than the maximum tolerated dose of capecitabine. Capecitabine is dosed at 135 mg/kg and tivozanib is dosed at 5 mg/kg. The following is depicted in the graph: vehicle (▪, Veh) and tivozanib+capecitabine (▴, cap135+tivo5).

FIG. 3B is a graph showing the results from an experiment to measure the tumor inhibitory activity of tivozanib+capecitabine (co-administered) with less than the maximum tolerated dose of tivozanib. Capecitabine is dosed at 269 mg/kg and tivozanib is dosed at 2 mg/kg. The following is depicted in the graph: vehicle (♦, Veh) and tivozanib+capecitabine (x, cap269+tivo2).

FIG. 4 are photographs of the tumor blood vessels showing the results from an experiment to measure the anti-angiogenesis activity of capecitabine (alone), tivozanib (alone) and tivozanib+capecitabine (co-administered). The capecitabine response (capecitabine 9d) is measured after 9 days, the tivozanib response (tivozanib 19d) is measured after 19 days. The co-administered capecitabine+tivozanib response (capecitabine+tivozanib 35d) is measured after 35 days.

FIG. 5A is a graph showing the results from an experiment to measure thymidine phosphorylase (TP) up-regulation in murine breast tumor BH216 following administration of tivozanib. Tivozanib is dosed at 5 mg/kg. Tumor samples were collected and analyzed by qRT-PCR after 0.5, 2, 6 and 16 hours.

FIG. 5B is a graph showing the results from an experiment to measure TP up-regulation in murine breast tumor BH225 following administration of tivozanib. Tivozanib is dosed at 5 mg/kg. Tumor samples were collected and analyzed by qRT-PCR after 6, 24 and 30 (6 hours after the second dose) hours.

FIGS. 5C-D are graphs showing the results from an experiment to measure TP up-regulation in two additional tumors, murine breast tumors (C) BH229 and (D) BH 281, following administration of tivozanib. Tivozanib is dosed at 5 mg/kg. Tumor samples were collected and analyzed by qRT-PCR after 6 and 8 hours.

FIGS. 6A-D are a series of graphs showing the results of an experiment to measure TP induction following tivozanib administration in (A) murine breast tumor BH216-derived primary culture, (B) a mouse monocyte cell line (MH-S), (C) human endothelial cells (HUVECs), and (D) a human monocyte cell line (U937). Tivozanib is added to the culture at the indicated concentrations. TP induction was observed only in the BH216 primary culture and not in the monocyte cell lines and cultured endothelial cells.

FIG. 7 is a graph showing the results of an experiment to measure TP induction in BH216 tumors following administration of tivozanib, 5 mg/kg, oral; antibody B20-4.1, 5 or 20 mg/kg, intravenous; sunitinib, 40 mg/kg, oral; sorafenib, 60 mg/kg, oral. **All test points with antibody B20-4.1 had a p value>0.05 except for antibody B20-4.1 dosed at 5 mg/kg (mpk) after 24 hours, which showed down-regulation.

### DETAILED DESCRIPTION

The invention relates to methods of (i) reducing tumor growth and (ii) increasing the efficacy and survival rates of patients suffering from solid tumor cancers, e.g., breast tumors and colorectal tumors. The disclosed methods are based on a combination therapy where tivozanib, a VEGF tyrosine kinase inhibitor, is administered with capecitabine, a pyrimidine antagonist. The disclosed methods surprisingly show that at least one of tivozanib and capecitabine may be administered at reduced dose when the two drugs are administered in combination. The disclosed methods also suggest that TP induction by tivozanib when used in combination with capecitabine results in synergistic anti-tumor activity.

### I. DEFINITIONS

For convenience, certain terms in the specification, examples, and appended claims are collected in this section.

As used herein, “combination therapy,” means co-administering tivozanib and capecitabine as part of a specific dosage regimen intended to provide the beneficial effect from the co-action of these therapeutic agents. Administration of these therapeutic agents in combination typically is carried out over a defined time period (e.g., weeks or months). Combination therapy includes administration of the therapeutic agents in a concurrent manner, e.g., the therapeutic agents can be administered at the same or a different time and can be administered by the same route or by different routes. Concurrent administration of each therapeutic agent can be effected by any appropriate route. For example, tivozanib and/or capecitabine can be administered orally.

As used herein, “pharmaceutically acceptable” or “pharmacologically acceptable” mean molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well-known in the art. Except insofar as any conventional media or agent is incompatible with the active ingredient, its use in the therapeutic compositions is contemplated. Supplementary active ingredients can also be incorporated into the compositions.

As used herein, “tivozanib” means N-{2-chloro-4-[(6,7-dimethoxy-4-quinolyl)oxy]-phenyl}-N′-(5-methyl-3-isoxazolyl)urea and having the following chemical structure:

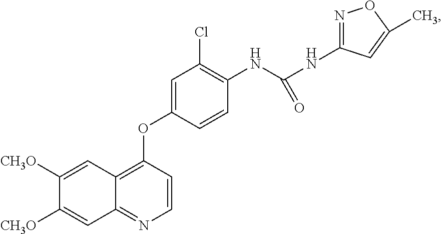

including pharmaceutically acceptable salts, solvates, solvates of a pharmaceutically acceptable salt, esters, or polymorphs thereof. See, for example, U.S. Pat. Nos. 6,821,987; 7,166,722; and 7,211,587, each of which are incorporated herein by reference in their entirety. In certain embodiments, tivozanib is N-{2-chloro-4-[(6,7-dimethoxy-4-quinolyl)oxy]-phenyl}-N′-(5-methyl-3-isoxazolyl)urea or hydrates of a hydrochloride salt. In certain embodiments, tivozanib is N-{2-chloro-4-[(6,7-dimethoxy-4-quinolyl)oxy]-phenyl}-N′-(5-methyl-3-isoxazolyl)urea monohydrochloric acid salt monohydrate as shown below:

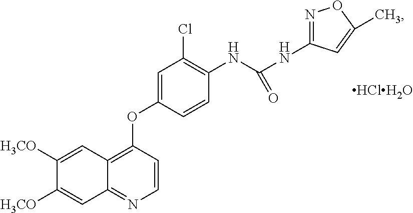

As used herein, “capecitabine” means 5′-deoxy-5-fluoro-N-[(pentyloxy)carbonyl]-cytidine, which has the structure shown below:

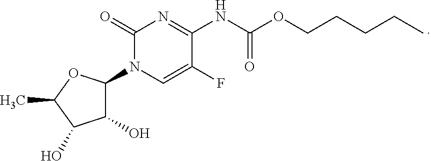

See, for example, U.S. Pat. Nos. 5,472,949 and 4,966,891, each of which are incorporated herein by reference in their entirety. Capecitabine (XELODA®) is available commercially from Roche.

As used herein, “5-fluorouracil” means 5-fluoro-2,4-(1H,3H)-pyrimidinedione, which has the structure shown below:

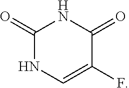

5-fluorouracil is also known as 5-FU. 5-Fluorouracil is available commercially from Bioniche Pharma.

### II. METHODS

The concurrent administration of tivozanib and capecitabine can be by concurrent administration of separate formulations, i.e., a tivozanib formulation and a capecitabine formulation. As disclosed herein, administration of separate formulations is “concurrent” if the timing of their administration is such that the pharmacological activities of tivozanib and capecitabine overlap in time, thereby exerting a combined antitumor effect in the patient. In the context of the present invention, concurrent administration, i.e., combination therapy, does not require the two drugs to be administered simultaneously, nor to be administered by the same route of administration, nor to be present in the body of the patient at the same time. The temporal overlap of the pharmacological activities of tivozanib and capecitabine will depend on factors including (with respect to each of the two drugs): dosage, frequency and timing of administration, half-life, and pharmacokinetics.

The half-life of tivozanib in the human body is in the range of 3.8-4.7 days. Tivozanib accumulates in the serum after chronic dosing to a degree that would be expected based on its half-life. Serum levels reach steady state after about 2-3 weeks.

The elimination half-life of capecitabine in the human body is approximately ¾ of an hour. The primary metabolite of capecitabine, i.e., fluorouracil, has DNA synthesis inhibitor activity and has the same approximate half-life of the parent compound. The metabolites of capecitabine appear to accumulate more than would be expected based on their half lives, for example, the area under the curve (AUC) of fluorouracil was 35% higher at day 14 than day 1.

It is contemplated herein that 5-fluorouracil (5-FU) may be used as an alternative to capecitabine.

### III. DOSAGE

When tivozanib and capecitabine are administered concurrently the dosage of tivozanib may be 0.5-2 mg, 0.5-1.5 mg, 1.0-2.0 mg, 1.0-1.5 mg or 1.4-1.6 mg per day. Alternatively, when tivozanib and capecitabine are concurrently administered, the dosage of tivozanib may be 0.5-0.6 mg, 0.6-0.7 mg, 0.7-0.8 mg, 0.8-0.9 mg, 0.9-1.0 mg, 1.0-1.1 mg, 1.1-1.2 mg, 1.2-1.3 mg, 1.3-1.4 mg, 1.4-1.5 mg, 1.5-1.6 mg, 1.6-1.7 mg, 1.7-1.8 mg, 1.8-1.9 mg or 1.9-2.0 mg daily. In one embodiment, the dosage of tivozanib may be 1.5 mg daily.

When tivozanib and capecitabine are administered concurrently the dosage of capecitabine may be 400-2000 mg/m², 400-1600 mg/m², 400-1250 mg/m², 750-1250 mg/m², 600-1300 mg/m² or 700-1300 mg/m² twice daily. Alternatively, when tivozanib and capecitabine are concurrently administered, the dosage of capecitabine may be 400-500 mg/m², 500-600 mg/m², 600-700 mg/m², 700-800 mg/m², 800-900 mg/m², 900-1000 mg/m², 1000-1100 mg/m², 1100-1200 mg/m², 1200-1300 mg/m², 1300-1400 mg/m², 1400-1500 mg/m² 1500-1600 mg/m², 1600-1700 mg/m², 1700-1800 mg/m², 1800-1900 mg/m² or 1900-2000 mg/m² twice daily. In one embodiment, the dosage of capecitabine may be 1250 mg/m² twice daily. In another embodiment, the dosage of capecitabine may be 800 mg/m² twice daily. In another embodiment, the dosage of capecitabine may be 2000 mg/m² twice daily.

When tivozanib and 5-fluorouracil are administered concurrently the dosage of 5-fluorouracil may be 2-20 mg/kg, 2-12 mg/kg or 6-12 mg/kg once daily. Alternatively, when tivozanib and 5-fluorouracil are concurrently administered, the dosage of 5-fluorouracil may be 2-4 mg/kg, 4-6 mg/kg, 6-8 mg/kg, 8-10 mg/kg, 10-12 mg/kg, 12-14 mg/kg, 14-16 mg/kg, 16-18 mg/kg or 18-20 mg/kg daily. In one embodiment, the dosage of 5-fluorouracil may be 12 mg/kg daily. Alternatively, the dosage of 5-fluorouracil may be 6 mg/kg daily.

It is contemplated herein for any one of the tivozanib dosages that the skilled person can select any one of the capecitabine dosages. For example, a tivozanib dosage of 1.5 mg daily could be combined with a capecitabine dosage of 1250 mg/m² twice daily. Alternatively, a tivozanib dosage of 1.2 mg daily could be combined with a capecitabine dosage of 1250 mg/m² twice daily. In another embodiment, a tivozanib dosage of 1.0 mg daily could be combined with a capecitabine dosage of 1250 mg/m² twice daily. Additionally, a tivozanib dosage of 0.8 mg daily could be combined with a capecitabine dosage of 1250 mg/m² twice daily. Alternatively, a tivozanib dosage of 0.5 mg daily could be combined with a capecitabine dosage of 1250 mg/m² twice daily. For example, a tivozanib dosage of 1.5 mg daily could be combined with a capecitabine dosage of 800 mg/m² twice daily. Alternatively, a tivozanib dosage of 1.2 mg daily could be combined with a capecitabine dosage of 800 mg/m² twice daily. In another embodiment, a tivozanib dosage of 1.0 mg daily could be combined with a capecitabine dosage of 800 mg/m² twice daily. Additionally, a tivozanib dosage of 0.8 mg daily could be combined with a capecitabine dosage of 800 mg/m² twice daily. Alternatively, a tivozanib dosage of 0.5 mg daily could be combined with a capecitabine dosage of 800 mg/m² twice daily. For example, a tivozanib dosage of 1.5 mg daily could be combined with a capecitabine dosage of 600 mg/m² twice daily. Alternatively, a tivozanib dosage of 1.2 mg daily could be combined with a capecitabine dosage of 600 mg/m² twice daily. In another embodiment, a tivozanib dosage of 1.0 mg daily could be combined with a capecitabine dosage of 600 mg/m² twice daily. Additionally, a tivozanib dosage of 0.8 mg daily could be combined with a capecitabine dosage of 600 mg/m² twice daily. Alternatively, a tivozanib dosage of 0.5 mg daily could be combined with a capecitabine dosage of 600 mg/m² twice daily. For example, a tivozanib dosage of 1.5 mg daily could be combined with a capecitabine dosage of 2000 mg/m² twice daily. Alternatively, a tivozanib dosage of 1.2 mg daily could be combined with a capecitabine dosage of 2000 mg/m² twice daily. In another embodiment, a tivozanib dosage of 1.0 mg daily could be combined with a capecitabine dosage of 2000 mg/m² twice daily. Additionally, a tivozanib dosage of 0.8 mg daily could be combined with a capecitabine dosage of 2000 mg/m² twice daily. Alternatively, a tivozanib dosage of 0.5 mg daily could be combined with a capecitabine dosage of 2000 mg/m² twice daily.

Alternatively, it is contemplated herein for any one of the tivozanib dosages that the skilled person can select any one of the 5-fluorouracil dosages. For example, a tivozanib dosage of 1.5 mg daily could be combined with a 5-fluorouracil dosage of 12 mg/kg daily. Alternatively, a tivozanib dosage of 1.2 mg daily could be combined with a 5-fluorouracil dosage of 12 mg/kg daily. Additionally, a tivozanib dosage of 0.8 mg daily could be combined with a 5-fluorouracil dosage of 12 mg/kg daily. In another embodiment, a tivozanib dosage of 0.5 mg daily could be combined with a 5-fluorouracil dosage of 12 mg/kg daily. For example, a tivozanib dosage of 1.5 mg daily could be combined with a 5-fluorouracil dosage of 6 mg/kg daily. Additionally, a tivozanib dosage of 1.2 mg daily could be combined with a 5-fluorouracil dosage of 6 mg/kg daily. Additionally, a tivozanib dosage of 0.8 mg daily could be combined with a 5-fluorouracil dosage of 6 mg/kg daily. In another embodiment, a tivozanib dosage of 0.5 mg daily could be combined with a 5-fluorouracil dosage of 6 mg/kg daily.

### IV. ADMINISTRATION

When tivozanib and capecitabine are administered concurrently, the drug combination can be administered in a single formulation or as separate formulations. Tivozanib may be administered as an oral tablet or capsule or as an intravenous (IV) infusion. When administered as an oral tablet or capsule, the dosage of tivozanib may be a single capsule or tablet or two or more capsules or tablets. Capecitabine may be administered as an oral tablet or capsule or as an IV infusion. When administered as an oral tablet or capsule, the dosage of capecitabine may be a single capsule or tablet or two or more capsules or tablets. In some embodiments, both tivozanib and capecitabine are administered as oral tablets or capsules. In other embodiments, both tivozanib and capecitabine are administered as IV infusions. Alternatively, tivozanib may be administered as an oral tablet or capsule and capecitabine may be administered as an IV infusion. Conversely, tivozanib may be administered as an IV infusion and capecitabine may be administered as an oral tablet or capsule.

Alternatively, when tivozanib and 5-fluorouracil are administered concurrently, the drug combination can be administered in a single formulation or as separate formulations. Tivozanib may be administered as an oral tablet or capsule or as an intravenous (IV) infusion. When administered as an oral tablet or capsule, the dosage of tivozanib may be a single capsule or tablet or two or more capsules or tablets. 5-Fluorouracil may be administered as a topical cream or as an IV infusion. In some embodiments, both tivozanib and 5-fluorouracil are administered as IV infusions. Alternatively, tivozanib may be administered as an oral tablet or capsule and 5-fluorouracil may be administered as an IV infusion. Conversely, tivozanib may be administered as an IV infusion and 5-fluorouracil may be administered as a topical cream.

Tivozanib may be administered on a repeating schedule of one dose (e.g., a single dosage contains 0.5-2.0 mg of tivozanib) per day for three weeks, followed by one week off (i.e., 3 weeks on, 1 week off). For example, tivozanib may be administered on a repeating schedule of 0.5-2 mg, 0.5-1.5 mg, 1.0-2.0 mg, 1.0-1.5 mg or 1.4-1.6 mg per day for three weeks, followed by one week off (i.e., 3 weeks on, 1 week off). In other embodiments, tivozanib may be administered one dose (e.g., a single dosage contains 0.5-2.0 mg of tivozanib) per day. For example, tivozanib may be administered 0.5-2 mg, 0.5-1.5 mg, 1.0-2.0 mg, 1.0-1.5 mg or 1.4-1.6 mg daily.

Capecitabine may be administered on a repeating schedule of one dose (e.g., a single dosage contains 400-1600 mg/m² of capecitabine) twice daily for two weeks, followed by one week off (i.e., 2 weeks on, 1 week off). For example, capecitabine may be administered on a repeating schedule 400-1600 mg/m², 400-1250 mg/m², 750-1250 mg/m², 600-1300 mg/m², 700-1300 mg/m² or 1100-1200 mg/m² twice daily for two weeks, followed by one week off (i.e., 2 weeks on, 1 week off). Alternatively, capecitabine may be administered one dose (e.g., a single dosage contains 400-1600 mg/m² of capecitabine) twice daily. For example, capecitabine may be administered 400-1600 mg/m², 400-1250 mg/m², 750-1250 mg/m², 600-1300 mg/m², 700-1300 mg/m² or 1100-1200 mg/m² twice daily.

5-Fluorouracil may be administered on a dose of 5-20 mg daily for the first four days, followed by a dose of 2-10 mg every other day until the twelfth day. Alternatively, 5-fluorouracil may be administered one dose (e.g., a single dosage contains 2-20 mg of 5-fluorouracil) daily.

For example, tivozanib may be orally administered on a repeating schedule of a dosage of 0.5-2.0 mg daily for three weeks, followed by one week off, while capecitabine may be orally administered on a repeating schedule a dosage of 400-1600 mg/m² twice daily for two weeks, followed by one week off.

In another example, tivozanib may be orally administered on a repeating schedule of a dosage of 0.5-2.0 mg daily for three weeks, followed by one week off, while capecitabine may be orally administered on a repeating schedule of a dosage of 400-2000 mg/m² twice daily for one week, followed by one week off. In certain embodiments, the tivozanib and capecitabine regimens are initiated on the same day. Alternatively, the capecitabine regimen may be initiated later, e.g., 3, 5, 7, 9 days later, than tivozanib regimen. In certain embodiments, capecitabine (administered in combination with tivozanib) may be orally administered on a repeating schedule of a dosage of 2000 mg/m² twice daily for one week, followed by one week off.

In another example, tivozanib may be orally administered on a repeating schedule of a dosage of 0.5-2.0 mg daily for three weeks, followed by one week off, while 5-fluorouracil may be administered by IV infusion a dose of 5-20 mg daily for the first four days, followed by a dose of 2-10 mg every other day until the twelfth day.

Reduced dosages of tivozanib may reduce the associated side effects, e.g., reduced dosages may reduce one or more of the following side effects: hypertension and dysphonia. Reduced dosages of capecitabine may reduce the associated side effects, e.g., reduced dosages may reduce one or more of the following side effects: hand-and-foot syndrome (pain, swelling or redness of your hands or feet), diarrhea, nausea, vomiting, dehydration, sores in the mouth and throat, stomach area pain, tiredness or weakness, fever, and joint and muscle pain. Reduced dosages of tivozanib and/or capecitabine may reduce one or more of any of the above side effects by at least 10%, 20%, 30%, 40%, 50%, 60%, 70%, 80%, 90%, 95%, 98% or 100%.

The method of the invention is suitable for treating various types of solid tumors, e.g., breast tumors and colorectal tumors. The present invention is particularly suitable for treating breast tumors.

### EXAMPLES

The invention is further illustrated by the following examples. The following examples are provided for illustration purposes only, and are not to be construed as limiting the scope or content of the invention in any way.

### Example 1

### Inhibition of Tumor Cell Proliferation

The combination of tivozanib and capecitabine was evaluated in tumors that exhibited minor response to either capecitabine or tivozanib alone as single agents at clinically relevant doses.

The chimeric inducible breast tumor mouse models were generated as described in U.S. Pat. No. 7,622,630, which is incorporated herein by reference in its entirety. The Her2-driven breast tumor (BH) model used herein has the following genotype: MMTVrTTA, tetO HER2, INK4A(−/−), luciferase reporter.

When tumors reached approximately 200 mm³, the mice were randomized into 4 groups of 10 mice each. One group received vehicle control. Each of the other 3 groups were treated with one of the two agents either administered alone or in combination. Both agents were administered orally.

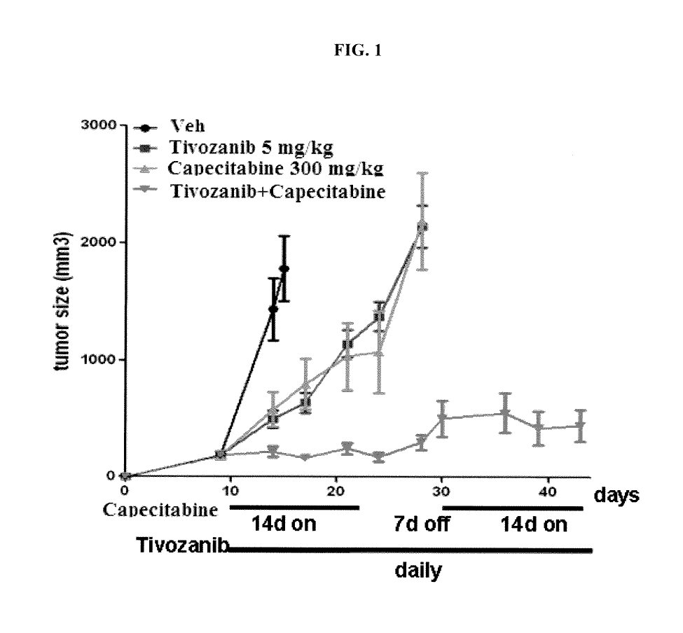

As shown in FIG. 1, an experiment was designed to evaluate the effect of the combination therapy on tumor size. Tivozanib was dosed at 5 mg/kg daily for more than 4 weeks. Capecitabine was dosed at 300 mg/kg daily for two weeks followed by a one week break (2 weeks on, 1 week off) for more than 4 weeks. In the mice receiving tivozanib alone or capecitabine alone there was only moderate reduction in tumor growth, but in the mice receiving the combination of the two agents, tumor stasis was observed.

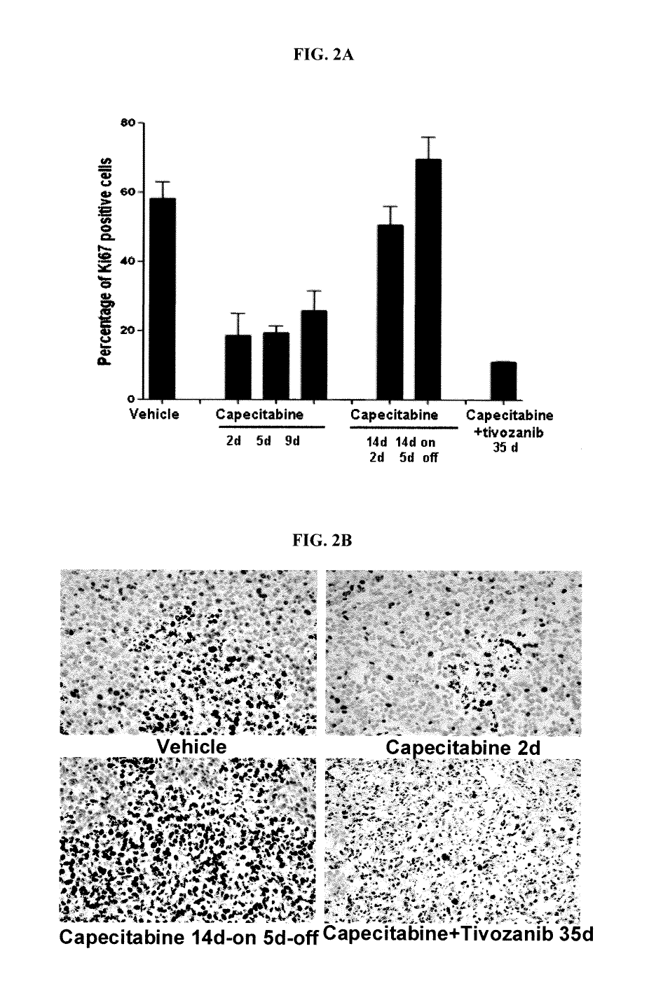

To determine the mechanism of action of the combination therapy, capecitabine was administered alone and in combination with tivozanib, over two dosage cycles (FIG. 2A). Capecitabine was dosed at 300 mg/kg daily for two weeks, followed by a seven day break, and then an additional two weeks of capecitabine at 300 mg/kg daily. Tivozanib was dosed at 5 mg/kg daily for 5 weeks. The capecitabine monotherapy reduced tumor cell proliferation in the first two weeks, but between capecitabine cycles (during break periods), tumor cell proliferation rebound was observed. The combination of capecitabine and tivozanib achieved a near complete blockade of tumor cell proliferation as observed by Ki67 immunohistochemistry (FIG. 2B).

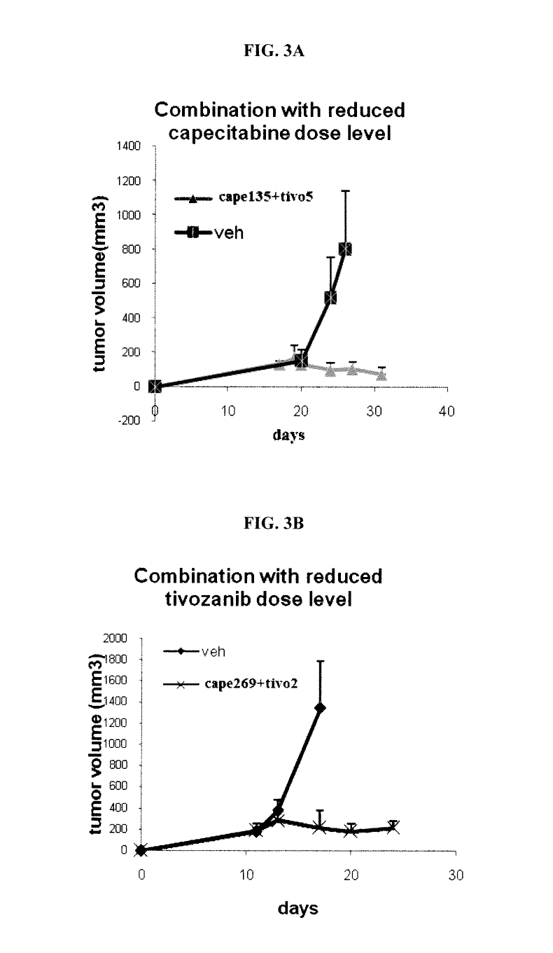

The synergistic effects of the combination therapy were evaluated utilizing two experiments with reduced dosages of capecitabine and tivozanib, respectively. The combination of tivozanib dosed at 5 mg/kg daily and capecitabine dosed at 135 mg/kg daily (reduced dose) for two weeks (FIG. 3A). Similarly, the combination of tivozanib dosed at 2 mg/kg daily (reduced dose) and capecitabine dosed at 300 mg/kg daily for two weeks (FIG. 3B). When either capecitabine was reduced from 300 mg/kg to 135 mg/kg or tivozanib was reduced from 5 mg/kg to 2 mg/kg, similar antitumor activity was achieved in the same tumor model, suggesting synergistic mechanisms.

Combination therapy of capecitabine and tivozanib resulted in complete tumor growth inhibition accompanied by extensive necrosis. This increased efficacy was achieved even at reduced doses for either agent, indicating synergistic mechanisms.

### Example 2

### Inhibition of Angiogenesis

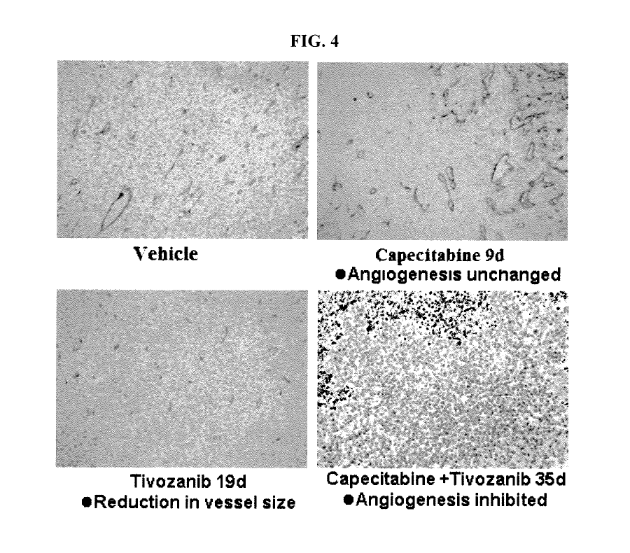

An experiment was designed to evaluate the effect of the combination therapy on angiogenesis (FIG. 4). Tivozanib was dosed at 5 mg/kg daily for 5 weeks. Capecitabine was dosed at 300 mg/kg daily for two weeks, followed by a seven day break, and then an additional two weeks of capecitabine at 300 mg/kg daily. In the mice receiving capecitabine alone there was no change in the angiogenesis profile, and in mice dosed with tivozanib alone there was only a reduction in vessel size but not microvessel density defined by vessel number per area. In mice receiving the combination of the two agents, angiogenesis was inhibited.

### Example 3

### Induction of Thymidine Phosphorylase (TP) in Tumors by Tivozanib

Thymidine phosphorylase is a key enzyme found in human tumors that converts capecitabine to the active form 5-FU. The enzyme is found at higher concentrations in most tumors compared to normal tissues, resulting in preferential release of 5-FU at the tumor site. It has been reported that in human xenograft tumors in mice, oral administration of capecitabine yields 100-200-fold higher levels of 5-FU AUC in tumors than in plasma. An experiment was designed to evaluate whether TP expression was elevated following tivozanib treatment.

Murine breast tumors (BH216, BH225, BH229 and BH281) were introduced subcutaneously into nude mice. For each tumor type, when tumors reached approximately 250 mm³, the mice were randomized into five groups, with three mice in each group. One group received vehicle (0.5% methyl-cellulose) and the remaining groups each received one dose of 5 mg/kg tivozanib. The mice were sacrificed and tumors collected at 0.5, 2, 6 and 16 hours post-dose. RNA was extracted from the tumors and TP expression was analyzed with qRT-PCR.

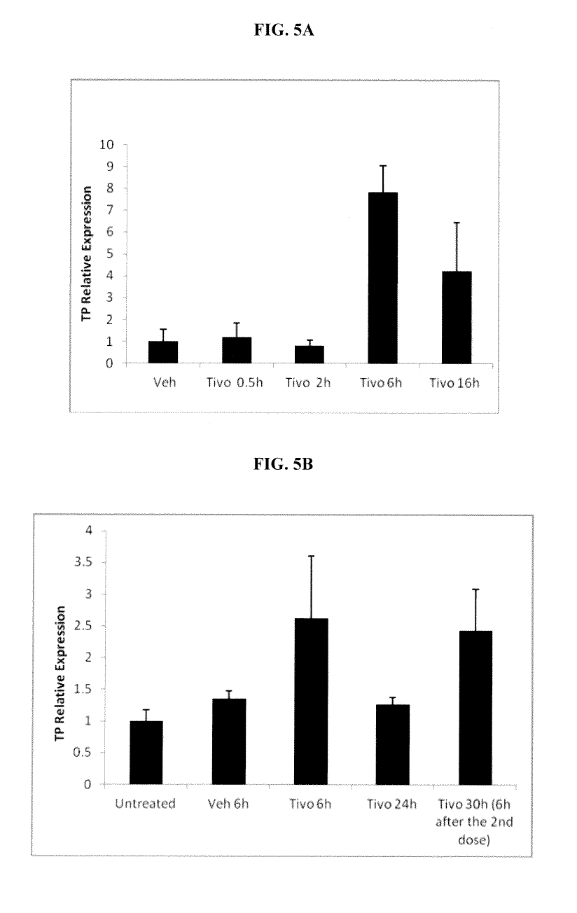

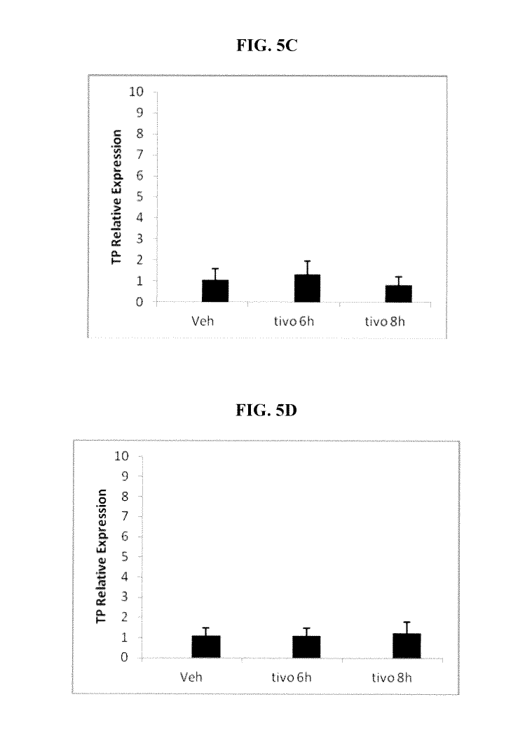

In tumors where a synergistic effect was observed when tivozanib and capecitabine were administered in combination, TP expression was significantly up-regulated following tivozanib treatment. As shown in FIG. 5A, an approximately 8-fold up-regulation of TP was observed 6 hours after tivozanib treatment in BH216 tumors. A significant elevation in TP expression was also observed 16 hours post-dose. Similar TP induction was also observed in a second tumor (BH225) (FIG. 5B). By contrast, no TP up-regulation was detected in 2 tumors (BH229 and BH281) where no synergistic antitumor effects were observed (FIG. 5C).

These results demonstrate that in tumors where synergistic effects were observed in combination with capecitabine, TP expression was elevated by tivozanib treatment.

### Example 4

### TP Up-Regulation is Observed when Tivozanib and Capecitabine are Concurrently Administered in Tumor Bearing Mice

This experiment examined whether tivozanib induces TP up-regulation when administered concurrently with capecitabine. Mice bearing BH216 tumors, which exhibited synergistic response to the combination treatment, were given one dose of tivozanib 5 mg/kg and one dose of capecitabine 269 mg/kg at the same time. Tumors were collected 3, 6, 16 and 24 hours post-dose (5 in each group) followed by RNA extraction and qRT-PCR analysis.

Comparable TP up-regulation was observed in the presence of capecitabine, as described in Example 3, indicating that there is no antagonistic drug-drug interaction between tivozanib and capecitibine when measuring TP induction.

### Example 5

### Tumors Cells are the Likely Origin of TP Up-Regulation

Tumor associated macrophages have been reported to express TP. The primary molecular target of tivozanib, VEGFR2, is primarily expressed in endothelial cells. This experiment was designed to determine which tumor cell-types induce TP expression following tivozanib treatment.

Tumor cells from tumor BH216, which exhibited a synergistic response to the combination treatment, were collected from untreated tumors and cultured in vitro. A mouse macrophage cell line (MH-S, ATCC Number: CRL-2019™) and a human monocyte cell line (U937, ATCC Number: CRL1593.2™) were used as surrogates of tumor associated macrophages (Davies et al., 2005, Nutr. 135:2651-2656; Koren et al., 1979, Nature 279:328-31).

Primary human umbilical vein endothelial cells (HUVEC, ATCC PCS-100-010), which express highly selective targets for tivozanib VEGFR2 and VEGFR3, were cultured under conventional conditions for endothelial cells (Nakamura et al., 2006, Cancer Res. 66:9134-9142).

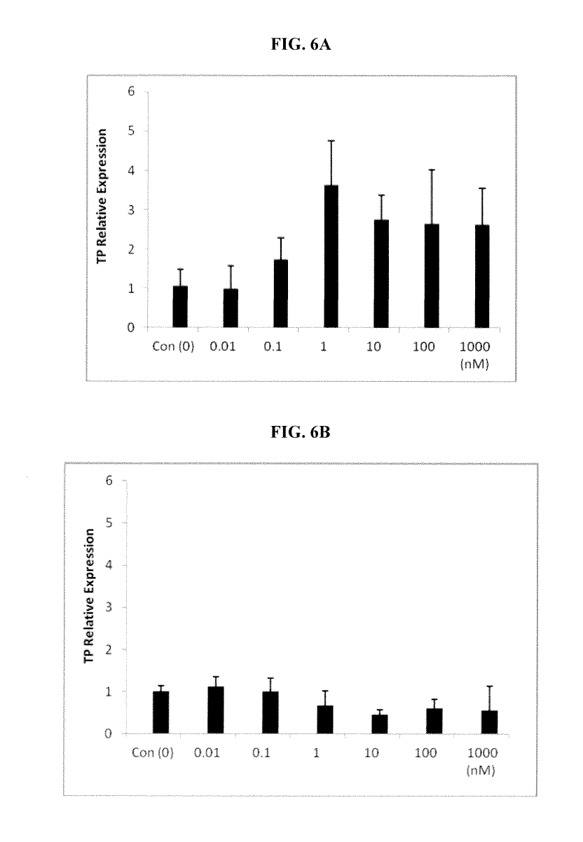

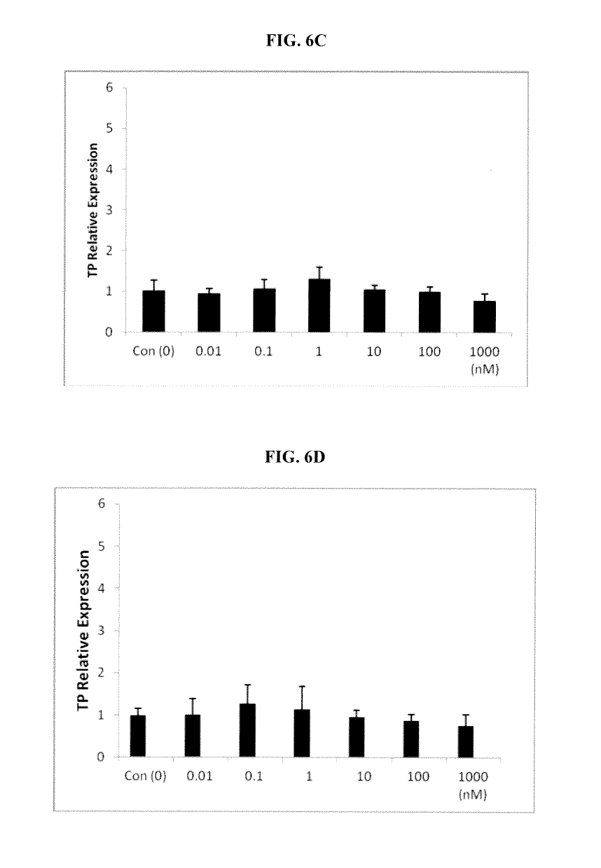

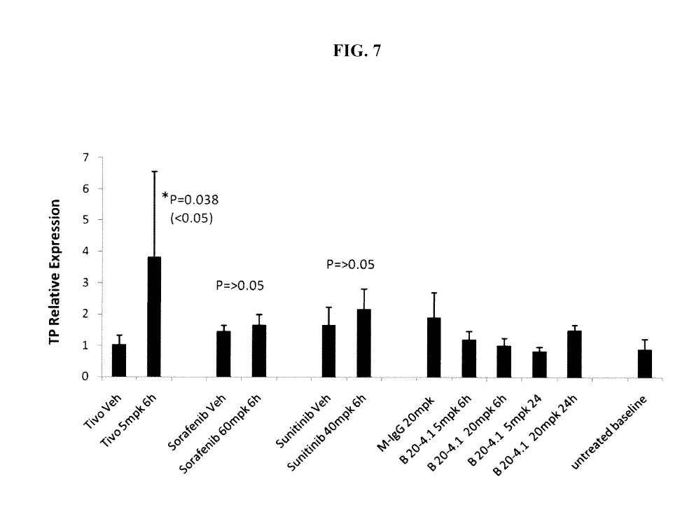

Each of the cultured cells were exposed to tivozanib at concentrations of 0.01, 0.1, 1.0, 10, 100 and 1000 nM for 3 hours, and then collected and subjected to RNA extraction and qRT-PCR analysis. TP induction was observed only in cultured BH216 tumor cells, not in the monocyte cell lines or HUVEC (FIGS. 6A-D). This suggests a likely tumor cell origin of TP induction observed in the tumors in vivo.

### Example 6

### Tivozanib but not Anti-VEGF Antibodies or Other VEGFR Inhibitors Induces TP Expression

Three VEGF pathway inhibitors, B20-4.1 (a VEGF antibody that is considered to be an equivalent of bevacizumab, but cross-reacts with mouse VEGF-A (Liang et al., 2006, J. Biol. Chem. 281:951-961) and two VEGF receptor inhibitors, sunitinib and sorafenib (Ivy et al., 2009 Nat. Rev. Clin. Oncology 6:569-579), were tested in comparison to tivozanib for the ability to induce TP expression. BH216 tumors were established subcutaneously as described above. When tumors reached approximately 300 mm³, mice were randomized into groups of five mice each. Each group received one dose of one of the following: vehicle (0.5% methyl-cellulose); tivozanib, 5 mg/kg, oral; antibody B20-4.1, 5 mg/kg or 20 mg/kg, intravenously; sunitinib, 40 mg/kg, oral; or sorafenib, 60 mg/kg, oral. Each of the VEGF inhibitors were administered at or about their respective maximum tolerated dosages. Tumors were collected after 6 hours for each of the tested inhibitors (as well as after 24 hours for antibody B20-4.1) and subjected to RNA extraction and qRT-PCR analysis.

As shown in FIG. 7, significant TP induction was observed only in tivozanib treated tumors (p<0.05), and not in tumors treated with other VEGF inhibitors. These data suggest that TP induction is specific to tivozanib when compared to other VEGF inhibitors. These data also suggest that TP induction by tivozanib when used in combination with capecitabine results in a synergistic anti-tumor activity.

### INCORPORATION BY REFERENCE

The entire disclosure of each of the patent documents and scientific articles cited herein is incorporated by reference for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

1. A method of treating a tumor in a subject, comprising co-administering to the patient: (a) tivozanib and (b) capecitabine.

2. The method of claim 1, wherein the subject is a human.

3. The method of claim 1, wherein tivozanib is dosed at 0.5-2.0 mg daily.

4. The method of claim 1, wherein tivozanib is dosed at 1.5 mg daily.

5. The method of claim 1, wherein capecitabine is dosed at 400-2000 mg/m² twice daily.

6. The method of claim 1, wherein capecitabine is dosed at 800 mg/m² twice daily.

7. The method of claim 1, wherein capecitabine is dosed at 1250 mg/m² twice daily.

8. The method of claim 1, wherein capecitabine is dosed at 2000 mg/m² twice daily.

9. The method of claim 1, wherein tivozanib is administered orally one dose per day for three weeks, followed by one week without tivozanib administration.

10. The method of claim 1, wherein capecitabine is administered orally on a repeating schedule of one dose twice daily for two weeks, followed by one week without capecitabine administration.

11. The method of claim 1, wherein capecitabine is administered orally on a repeating schedule of one dose twice daily for one week, followed by one week without capecitabine administration.

12. The method of claim 1, wherein the tumor is a breast tumor.

13. The method of claim 1, wherein the tumor is a colorectal tumor.

14. The method of claim 1, wherein tivozanib is N-{2-chloro-4-[(6,7-dimethoxy-4-quinolyl)oxy]-phenyl}-N′-(5-methyl-3-isoxazolyl)urea monohydrochloric acid salt monohydrate.
